(19) United States
(12) Patent Application Publication
(10) Pub. No.: US 2026/0123944 A1
(43) Pub. Date: May 7, 2026

(54) DEVICES FOR CROSSING OCCLUSIONS

(71) Applicant: ReFlow Medical, Inc., San Clemente, CA (US)

(72) Inventors: Thanh NGUYEN, Westminster, CA (US); Outhit BOUASAYSY, Corona, CA (US)

(21) Appl. No.: 19/370,421

(22) Filed: Oct. 27, 2025

Related U.S. Application Data
(60) Provisional application No. 63/714,435, filed on Oct. 31, 2024.

(51) Int. Cl. A61B 17/22; A61B 17/00

(52) U.S. Cl. CPC A61B 17/22; A61B 2017/00367; A61B 2017/22077; A61B 2017/22094

(57) ABSTRACT

Ablation, crossing, and cutting systems, devices and associated methods can relate to a crossing device that includes, in combination a spring-enhanced handle and body configured to controllably extend and retract an interventional tool within a microcatheter for addressing an occlusion within a vessel of a patient to provide improved access for supplemental treatment and reperfusion.

### CROSS-REFERENCE TO RELATED APPLICATION

This application claims the benefit of U.S. Provisional Application No. 63/714435, entitled “DEVICES FOR CROSSING OCCLUSIONS,” filed October 31, 2024, the entirety of which is incorporated herein by reference.

### TECHNICAL FIELD

The present description relates in general to medical devices, and more particularly to, for example, without limitation, devices for crossing occlusions.

### BACKGROUND

Chronic total occlusions (“CTO”) are vascular lesions characterized by heavy atherosclerotic plaque within the blood vessel, resulting in complete (or nearly complete) obstruction of blood flow across the lesion. Such occlusions can occur anywhere in a patient's vascular system. Since most lesions form gradually over a long period of time, the ischemic tissue downstream of the lesion has time to form collateral circulation. For example, in the case of coronary arteries, collateral vessels can form from the proximal artery and connect into the distal artery (“ipsilateral collaterals”), or collateral vessels can form from the other major arterial branches and connect into the distal artery (“contralateral collaterals”). When the lesion finally becomes a total occlusion, the collateral circulation is typically sufficient to keep the distal tissue alive, though ischemic. Accordingly, it is desirable to reestablish blood flow through or around the blockage in blood vessels by crossing the CTO and advancing therapeutic devices, such as a balloon angioplasty catheter, to dilate and treat the CTO. Likewise, in some cases it may be necessary to cross a CTO to gain access to a location along the vasculature distal to the CTO. CTOs can be more difficult to cross than partially occluded lesions because, rather than navigate a pre-existing lumen, a guidewire must either penetrate the lesion or, when penetrating the occlusion is impractically difficult and/or complicated, go around the lesion via a sub-intimal layer of a vessel wall.

The description provided in the background section should not be assumed to be prior art merely because it is mentioned in or associated with the background section. The background section may include information that describes one or more aspects of the subject technology.

### BRIEF DESCRIPTION OF THE DRAWINGS

FIG. 1 illustrates a perspective view of a crossing device in a retracted configuration, in accordance with one or more embodiments of the present disclosure.

FIG. 2 illustrates a perspective view of a crossing device in an extended configuration, in accordance with one or more embodiments of the present disclosure.

FIG. 3 illustrates a side view of a crossing device in a retracted configuration, in accordance with one or more embodiments of the present disclosure.

FIG. 4 illustrates a side view of a crossing device in an extended configuration, in accordance with one or more embodiments of the present disclosure.

FIG. 5 illustrates an exploded view of a crossing device, in accordance with one or more embodiments of the present disclosure.

FIG. 6 illustrates a cross-sectional view of a portion of a crossing device, in accordance with one or more embodiments of the present disclosure.

FIG. 7 illustrates a perspective view of an interventional tool of a crossing device, in accordance with one or more embodiments of the present disclosure.

FIG. 8 illustrates an exploded view of a crossing device, in accordance with one or more embodiments of the present disclosure.

FIG. 9 illustrates a sectional view of a portion of a crossing device in a retracted configuration, in accordance with one or more embodiments of the present disclosure.

FIG. 10 illustrates a sectional view of a portion of a crossing device in a retracted configuration, in accordance with one or more embodiments of the present disclosure.

FIG. 11 illustrates a partial sectional and exploded view of a portion of a crossing device, in accordance with one or more embodiments of the present disclosure.

FIG. 12 illustrates a side view of a crossing device in a retracted configuration, in accordance with one or more embodiments of the present disclosure.

FIG. 13 illustrates a side view of the crossing device of FIG. 12 in an extended configuration, in accordance with one or more embodiments of the present disclosure.

FIG. 14 illustrates a side view of the crossing device of FIGS. 12 and 13 in a locked configuration, in accordance with one or more embodiments of the present disclosure.

FIG. 15 illustrates an exploded perspective view of a portion of a crossing device, in accordance with one or more embodiments of the present disclosure.

FIG. 16 illustrates a side view of a crossing device in a retracted (e.g., proximal) configuration, in accordance with one or more embodiments of the present disclosure.

FIG. 17 illustrates a side view of the crossing device of FIG. 16 in a first (e.g., intermediate) locked configuration, in accordance with one or more embodiments of the present disclosure.

FIG. 18 illustrates a side view of the crossing device of FIGS. 16 and 17 in a second (e.g., distal) locked configuration, in accordance with one or more embodiments of the present disclosure.

FIG. 19 illustrates a side view of a portion of a portion of a crossing device, in accordance with one or more embodiments of the present disclosure.

FIG. 20 illustrates a side exploded view of the portion of the crossing device of FIG. 19, in accordance with one or more embodiments of the present disclosure.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

This disclosure relates to systems and apparatus for treating humans via allopathic or surgical intervention, minimally invasive surgical practices, endovascular procedures, percutaneous procedures, and related medical procedures. Specifically, this disclosure relates to systems for addressing vessel lumen-based issues and treatment of occlusions within a body, including those related to peripheral vascular disease states, cardiovascular diseases, cerebrovascular diseases, and others).

The devices and methods discussed herein may be employed for medical treatment and in conjunction with other devices and methods for medical treatment, as known to those skilled in the art. Improved ablation, crossing, and cutting devices and associated systems and methods are disclosed for treatment of occlusions within a body, owing to the paucity of effective devices available clinically, and the longstanding needs in this field of art.

Where a patient is indicated for certain treatment, monitoring, or intervention or suffers from a disease, a lumen of a blood vessel or other fluid-bearing vessel may become occluded. For example, a chronic total occlusion (“CTO”) or other partial or total obstruction of a blood vessel may occur or be the result of a thrombus, an embolism, atherosclerosis, or other disease that results in a blockage, restriction, or occlusion of fluid flow within a body. Often, during procedures, situations arise requiring immediate intervention, such as to address a CTO.

Where treatment of an occlusion by endovascular procedure is desired, medical professionals may attempt endovascular surgical procedures. Endovascular procedures include angioplasty procedures, stent placement, various scope procedures, and plethoric diagnostic, intermediary and interlocutory clot addressing schemes, which may include temporarily or permanently emplaced devices.

An occlusion may include a fibrous cap, composed of lipids, endothelial cells, macrophages, smooth muscle cells, foam cells, connective tissue, and other vascular materials. An occlusion may include calcification, such that the occlusion becomes hardened. Under these conditions, various endovascular procedures including thrombectomy, angioplasty, stent placement, retrieval of an obstruction, and others may be complicated or prevented by the difficulty or inability to penetrate the occlusion. For example, such procedures may require at least partial penetration of an occlusion before the procedure may be commenced or completed. Often, establishing some degree of reperfusion is critical.

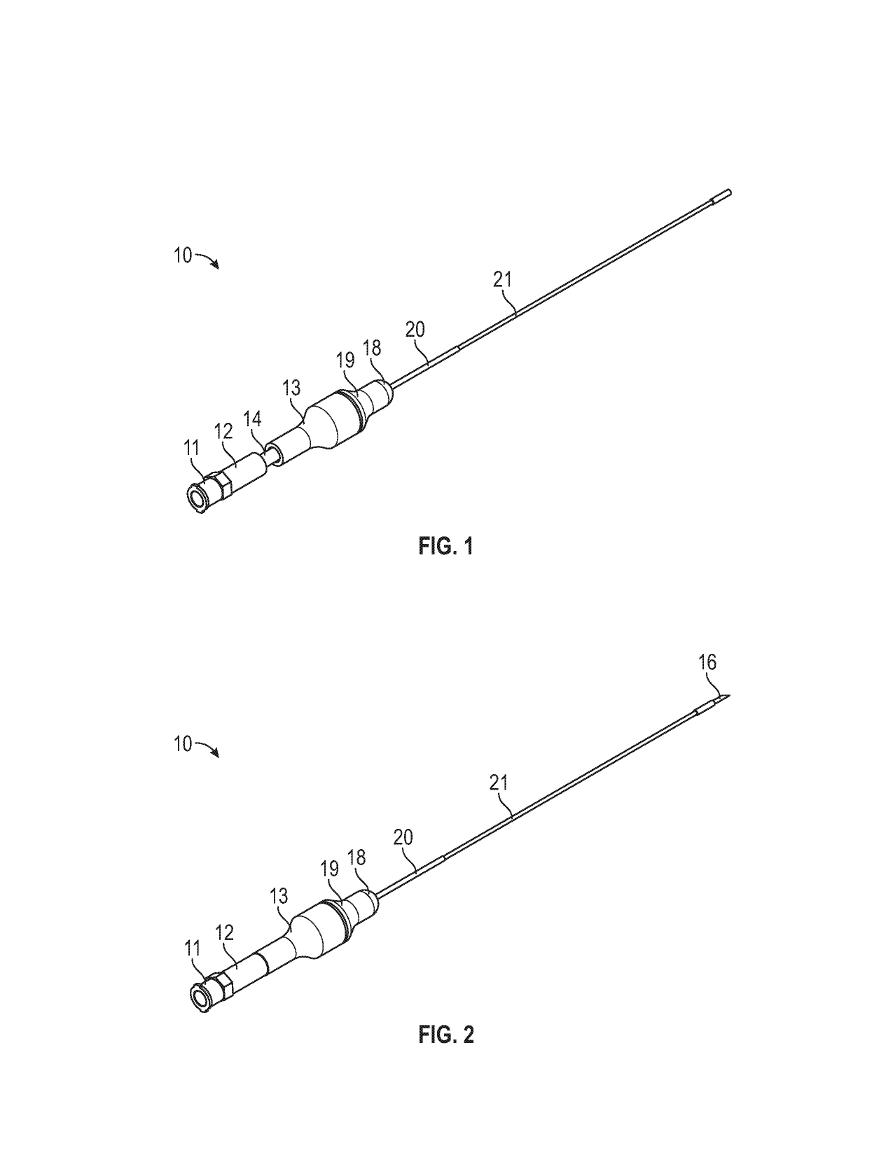

Certain details are set forth in the following description and FIGS. 1-20 to provide a thorough understanding of various embodiments of the disclosure. To avoid unnecessarily obscuring the description of the various embodiments of the disclosure, other details describing well-known structures and systems often associated with expandable structures, protruding features, and the components or devices associated with the manufacture of such structures are not set forth below. Moreover, many of the details and features shown in the figures are merely illustrative of particular embodiments of the disclosure. Accordingly, other embodiments can have other details and features without departing from the spirit and scope of the present disclosure. A person of ordinary skill in the relevant art will therefore understand that the present technology, which includes associated devices, systems, and procedures, may include other embodiments with additional elements or steps, and/or may include other embodiments without several of the features or steps shown and described below with reference to FIGS. 1–20. Furthermore, various embodiments of the disclosure can include structures other than those illustrated in the figures and are expressly not limited to the structures shown in the figures.

Referring to FIGS. 1-4, a crossing device can include features for facilitating a procedure with respect to an occlusion. As shown in FIG. 1, crossing device 10 may include proximal handle 13, catheter 21 (e.g., microcatheter), actuator 12, and interventional tool 16. In some embodiments, crossing device 10 allows for a handheld mechanical penetration of an occlusion. Surgeons and interventionalists have expressed ongoing needs for devices leveraging their “hand” or feel for manipulating devices in situ.

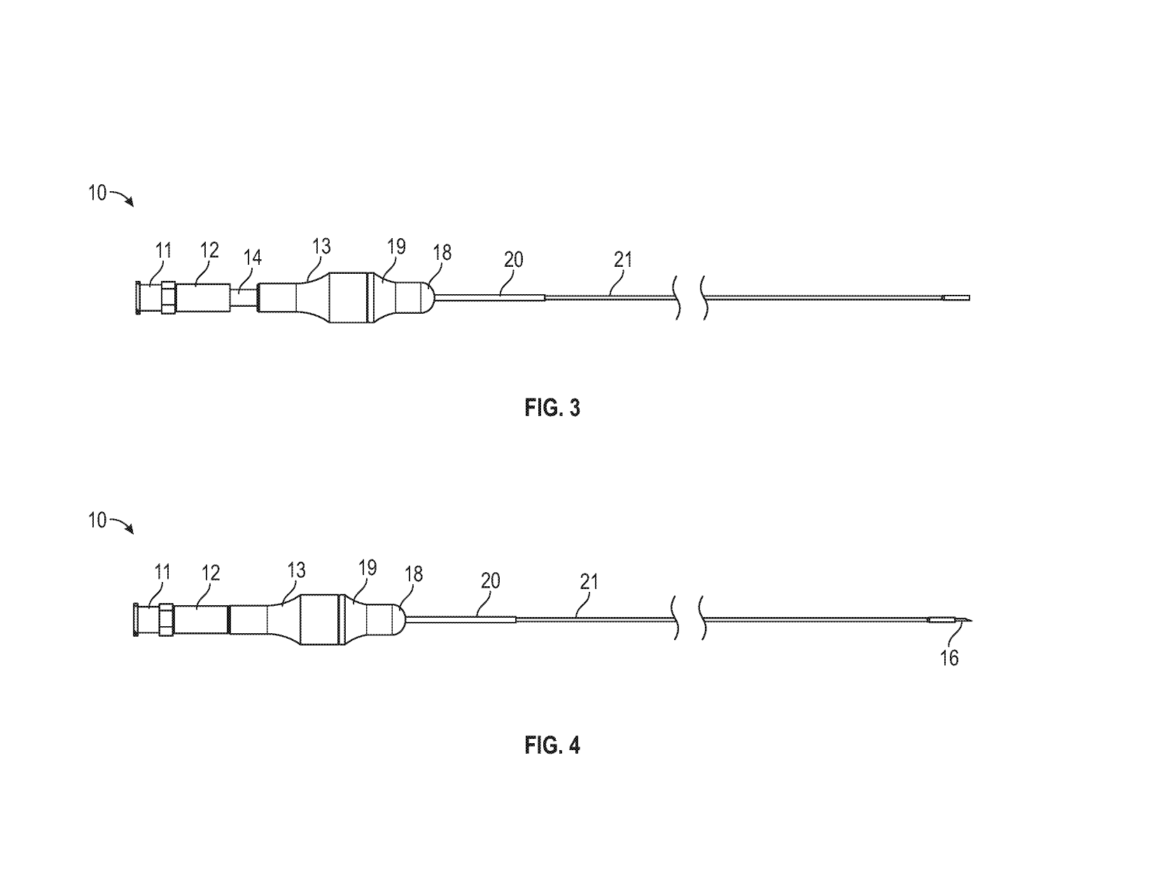

In some embodiments, proximal handle 13 may be attached to catheter 21, and actuator 12 may be attached to interventional tool 16, such that actuator 12 is moveable relative to proximal handle 13, and movement of actuator 12 relative to proximal handle 13 causes advancement and retraction of interventional tool 16 along the inner portion of catheter 21. FIGS. 1 and 3 show crossing device 10 in a retracted (e.g., natural or relaxed) position, with actuator 12 and interventional tool 16 retracted. FIGS. 2 and 4 show crossing device 10 in an extended (e.g., actuated) position, with actuator 12 and interventional tool 16 extended.

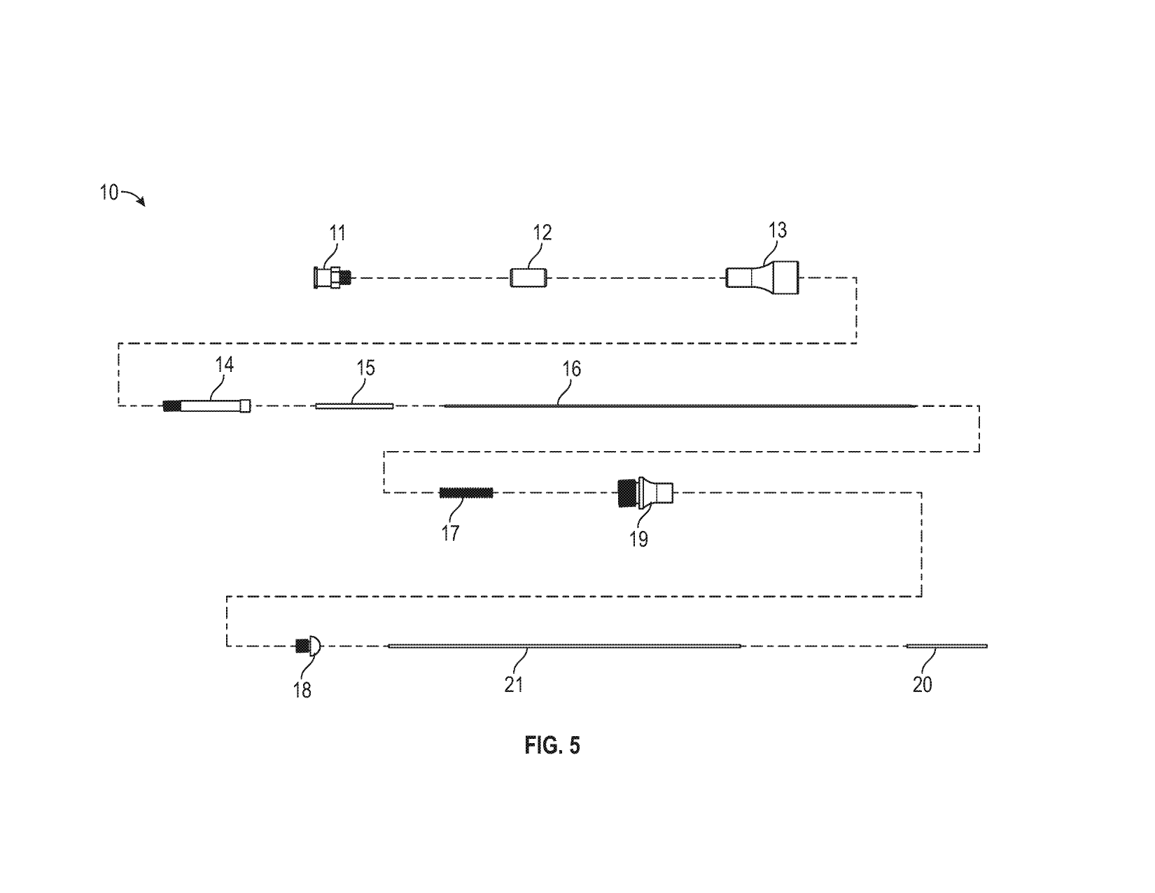

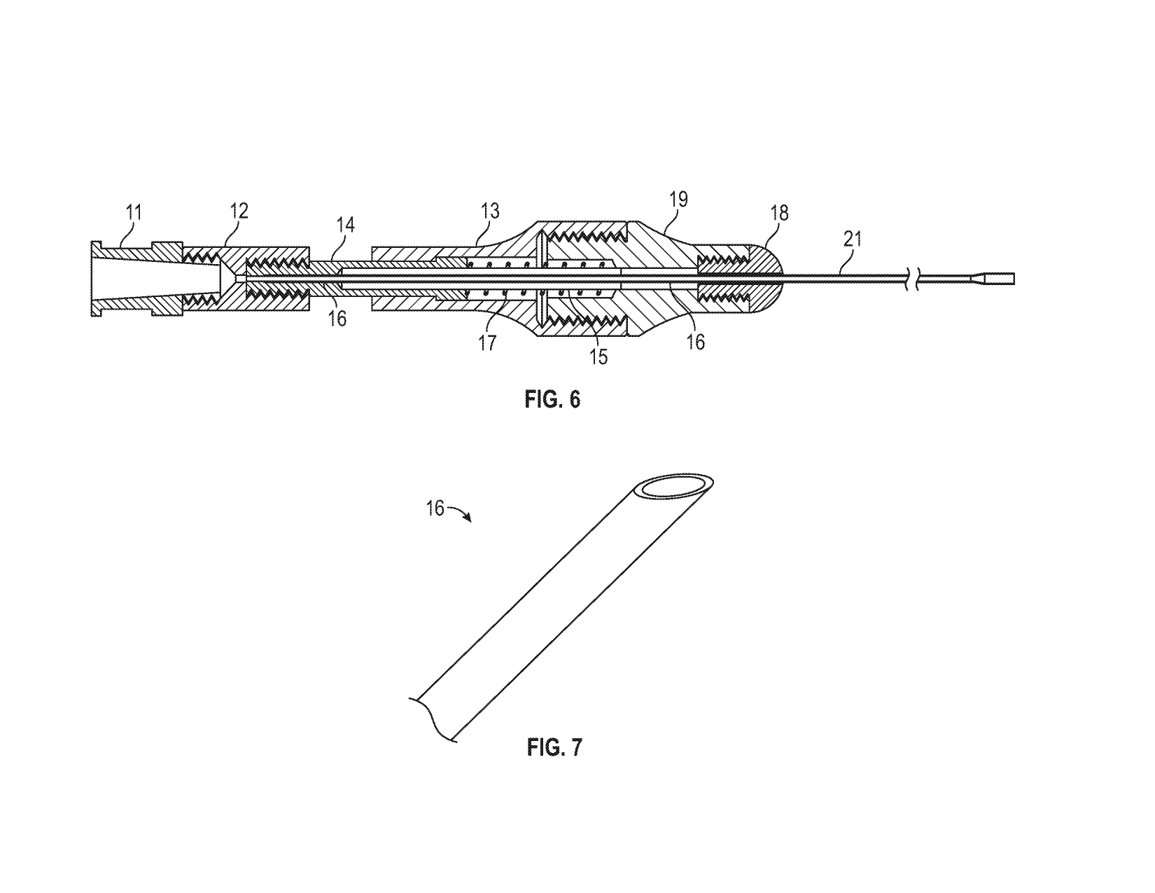

In some embodiments, actuator 12 may include or be combined with one or more of luer 11, plunger 14, hypo support 15, and interventional tool 16. For example, as shown in FIGS. 5 and 6, actuator 12, luer 11, plunger 14, hypo support 15, and interventional tool 16 may be combined to form an integrated unit. One or more interfaces (e.g., threading, etc.) or fixations (e.g., adhesive, epoxy, etc.) may be provided to combine said components.

In some embodiments, proximal handle 13 may include or be combined with one or more of distal handle 19, outer body 18, strain relief element 20, and catheter 21. For example, as shown in FIGS. 5 and 6, proximal handle 13, distal handle 19, outer body 18, strain relief element 20, and catheter 21 may be combined to form an integrated unit. One or more interfaces (e.g., threading, etc.) or fixations (e.g., adhesive, epoxy, etc.) may be provided to combine said components.

In some embodiments, spring 17 is disposed between at least a portion of distal handle 19 and at least a portion of plunger 14. For example, as shown in FIG. 6, spring 17 extends between plunger 14 and distal handle 19. In some embodiments, plunger 14 is attached to actuator 12, and outer body 18 and/or distal handle 19 are attached to proximal handle 13. Accordingly, spring 17 can provide a biasing force between actuator 12 and outer body 18.

In some embodiments, spring 17 may tend to separate actuator 12 from proximal handle 13 by way of elastic expansion. Actuator 12 and proximal handle 13 may be configured to set a limit on the separation. For example, as shown in FIGS. 5 and 6, plunger 14 may include a flange that contacts a portion (e.g., shoulder) of proximal handle 13 to prevent travel along the axis beyond a certain point.

In some embodiments, outer body 18 may be separate from and attachable to distal handle 19. Outer body 18 may be fixably attached to catheter 21 and removably attached to distal handle 19, such that a microcatheter may be exchanged by removing outer body 18. Such an option avoids the need to replace an entire system for need of a new microcatheter.

In some embodiments, crossing device 10 may facilitate use of a guidewire. Interventional tool 16 may be configured with a hollow inner portion to allow a guidewire to be advanced from a proximal end of interventional tool 16 to a distal end of interventional tool 16. A guidewire lumen within interventional tool 16 may be configured to accommodate any guidewire used for general vascular surgical procedures, such that a separate or distinct guidewire is not required for operation of crossing device 10.

In some embodiments, luer 11 may be configured to interface with actuator 12, as shown in FIGS. 5 and 6. Luer 11 may be a detachable hub/luer device configured to direct devices to catheter 21. Luer 11 may facilitate use of a support catheter, PTA balloon catheter, guidewire, or another lumen-configured or catheter-based supplemental treatment device to be exchanged with components of crossing device 10 during use, without removal of crossing device 10 from a patient. Such a configuration provides the ability to operate crossing device 10 in tandem or in sequence with other devices intended to deliver treatment to the same or nearby location.

In some embodiments, devices advanced within the lumen of interventional tool 16 or otherwise provided through luer 11 may be operated, manipulated, and moved either along with or independent of actuator 12, proximal handle 13, or their associated attached components.

In some embodiments, interventional tool 16 may be configured to penetrate an occlusion upon contact with the occlusion. The distal end of interventional tool 16 may include one of a variety of shapes to facilitate addressing the occlusion. An example is shown in FIG. 7. For example, interventional tool 16 may include an everted end, an end with a sharpened tip (e.g., a blade), a concave end, a convex end, a coring tip, or other geometry to address an occlusion. Interventional tool 16 may be hollow or solid. In some embodiments, interventional tool 16 includes a valve, closure, door, or other actuatable structure at a distal end portion thereof. For example, interventional tool 16 can include a valve (not shown) that transitions between open and closed configurations. By further example, the valve can selectively provide fluid communication between an inner lumen thereof (and/or luer 11) and an external environment based on the configuration of the valve. In some embodiments, valve can transition between the open and closed configurations based on a condition (e.g., pressure) and/or in response to a stimulus. For example, the valve can transition in response to actuation and/or other operation of another component of crossing device 10, such as a handle, plunger, and/or actuator thereof. In some embodiments, valve can transition between the open and closed configurations independent of other operations of crossing device 10.

In some embodiments, catheter 21 and interventional tool 16 may provide sufficient flexibility to provide trackability within vessels of a patient. Catheter 21 and interventional tool 16 may also provided sufficient rigidity to adequately transfer translational and rotational forces to be responsive at a distal end to a user located at a proximal end. In some embodiments, a tip at a distal end section of interventional tool 16 may be relatively rigid, to provide a force of impact to an occlusion upon extension of the same.

In some embodiments, interventional tool 16 may be configured to provide rotational motion at the point of deployment (e.g., at or near the distal end of crossing device 10). For example, as a user provides torque to actuator 12, the torque may be translated along interventional tool 16 to the distal end of interventional tool 16. A torque provided at the distal end may improve treatment of the occlusion. The torque may be applied and translated before, during, or after extension of interventional tool 16 beyond the distal end of catheter 21 and penetration of the occlusion.

In some embodiments, interventional tool 16 may be configured to automatically rotate as it advances longitudinally relative to catheter 21. For example, threading may be provided about the axis along which interventional tool 16 travels, such that travel of interventional tool 16 along the axis also results in rotation of interventional tool 16. For example, catheter 21 may be provided on at least a portion of its inner surface with threading, channels, or other guiding members to govern the manner in which interventional tool 16 is advanced and retracted therein. Interventional tool 16 may be provided with threading or other features to complement the guiding members of the axis along which interventional tool 16 travels. For example, at least a portion of interventional tool 16 may have a substantially spiral geometry adapted to interface with a complementary threading of catheter 21.

In some embodiments, interventional tool 16 may be configured to penetrate or cross a section of an occlusion. In some embodiments, at least a portion of the occlusion may be captured or enclosed by features of interventional tool 16. For example, ridges, protrusions, edges, and spiral geometries may be provided at the distal end of interventional tool 16 to capture or enclose at least a portion of the occlusion that is penetrated by interventional tool 16. Subsequently, interventional tool 16 may be retracted, whereby at least a portion of the occlusion is removed by interventional tool 16.

For example, where interventional tool 16 includes a substantially spiral geometry, interventional tool 16 may be advanced relative to catheter 21 with simultaneous longitudinal and rotational motion, whereby the spiral geometry captures at least a portion of the occlusion. Subsequently, interventional tool 16 may be retracted longitudinally, whereby the portions of the occlusion are maintained within the spiral geometry. For example, interventional tool 16 may be refracted with catheter 21. The foregoing may result in a path within the occlusion which may be the object of further operations, such as by the same device or others, or may be a path that at least partially restores a flow of blood and reperfusion through the occlusion.

In some embodiments, interventional tool 16 may be configured to be entirely or almost entirely disposed within catheter 21 at its distal end when crossing device 10 is in a retracted (e.g., natural or relaxed) position, as shown in FIGS. 1 and 3, and to be somewhat extended beyond the distal end of catheter 21 when crossing device 10 is in an extended (e.g., actuated) position, as shown in FIGS. 2 and 4.

In some embodiments, interventional tool 16 may be configured to lock relative to catheter 21 when in a retracted (e.g., natural or relaxed) position or an extended (e.g., actuated) position. Mechanisms to selectively or automatically lock and unlock interventional tool 16 may be provided and may be operable by a user located at a proximal end of crossing device 10 to activate or deactivate said mechanisms.

In some embodiments, crossing device 10 may be configured as either an “over the wire” device (see FIG. 6) or a “rapid exchange” device. For example, supplemental devices may be configured to interface with crossing device 10 in a rapid exchange configuration. Crossing device 10 may include at least one lumen to accommodate a supplemental device to be used in conjunction with other components of crossing device 10. For example, a rapid exchange lumen may provide entry and/or access at or near a proximal end of crossing device 10 and may extend to the distal end of crossing device 10 (e.g., the distal end of catheter 21).

In some embodiments, crossing device 10 may be used to address an occlusion. The distal end of crossing device 10, including a portion of catheter 21, interventional tool 16, or a guidewire, may be brought to the location of an occlusion. For example, the guidewire may first be brought to the occlusion, followed by catheter 21 and interventional tool 16. Interventional tool 16 may be extended beyond the distal end of catheter 21 by operation of actuator 12, as disclosed herein. As the occlusion is penetrated, increased advancement of catheter 21, interventional tool 16, or a guidewire may be provided. Increased advancement of catheter 21, interventional tool 16, or a guidewire may provide an improved position of the same to continue the crossing procedure in iterative steps. The process may be repeated in successive steps until the occlusion is crossed as desired.

In some embodiments, a method for causing interventional tool 16 to penetrate an occlusion is disclosed. In some embodiments, catheter 21 may be brought to an occlusion, such as a heavily calcified lesion or other obstruction, as discussed herein. Interventional tool 16 may be advanced by crossing device 10 such that it penetrates the occlusion. Interventional tool 16 may retract when actuator 12 is released, by the function of spring 17.

In some embodiments, the above-described steps may be repeated as desired. For example, the above-described steps may be repeated until the occlusion is entirely breached or until sufficient access is provided for other devices to act upon the occlusion. For example, a PTA balloon, stent device, or other catheter-based supplemental treatment device may operate within a channel created by operation of crossing device 10.

In some embodiments, the method and use of crossing device 10 may provide increased perfusion of fluid flow through the channel created by crossing device 10. The increased perfusion may provide improved conditions to facilitate breakdown of the occlusion, such that perfusion alone or in combination with other methods may remove threats presented by the occlusion.

In some embodiments, variations on embodiments may be made to provide customizable use and performance characteristics of crossing device 10. For example, spring 17 may be omitted such that actuator 12 may travel relative to proximal handle 13 along the axis within certain limitations, such that the user may selectively and manually advance and retract interventional tool 16 relative to catheter 21.

In some embodiments, crossing device 10 may be configured to allow interventional tool 16 to be in a retracted (e.g., natural or relaxed) position based on action taken by a user (not shown). The configuration shown in FIG. 6 may be modified such that spring 17 is located such that its tendency toward elastic expansion causes actuator 12 to be advanced toward proximal handle 13. In such a configuration, retraction of actuator 12 may compress spring 17, such that release of actuator 12 causes advancement of interventional tool 16 toward or beyond the distal end of catheter 21.

In some embodiments, crossing device 10 may include two springs to provide novel use and performance characteristics (not shown). For example, one spring may tend toward extension of interventional tool 16, and another spring may tend toward retraction of interventional tool 16. Interventional tool 16 may be configured such that the distal tip of interventional tool 16 is in a desired location when equilibrium between the two springs is achieved in a retracted (e.g., natural or relaxed) position. A user may selectively advance or retract interventional tool 16 by operation of actuator 12, and release for interventional tool 16 to allow release of any energy stored in the springs, with the interventional tool 16 eventually reaching equilibrium in a retracted (e.g., natural or relaxed) position.

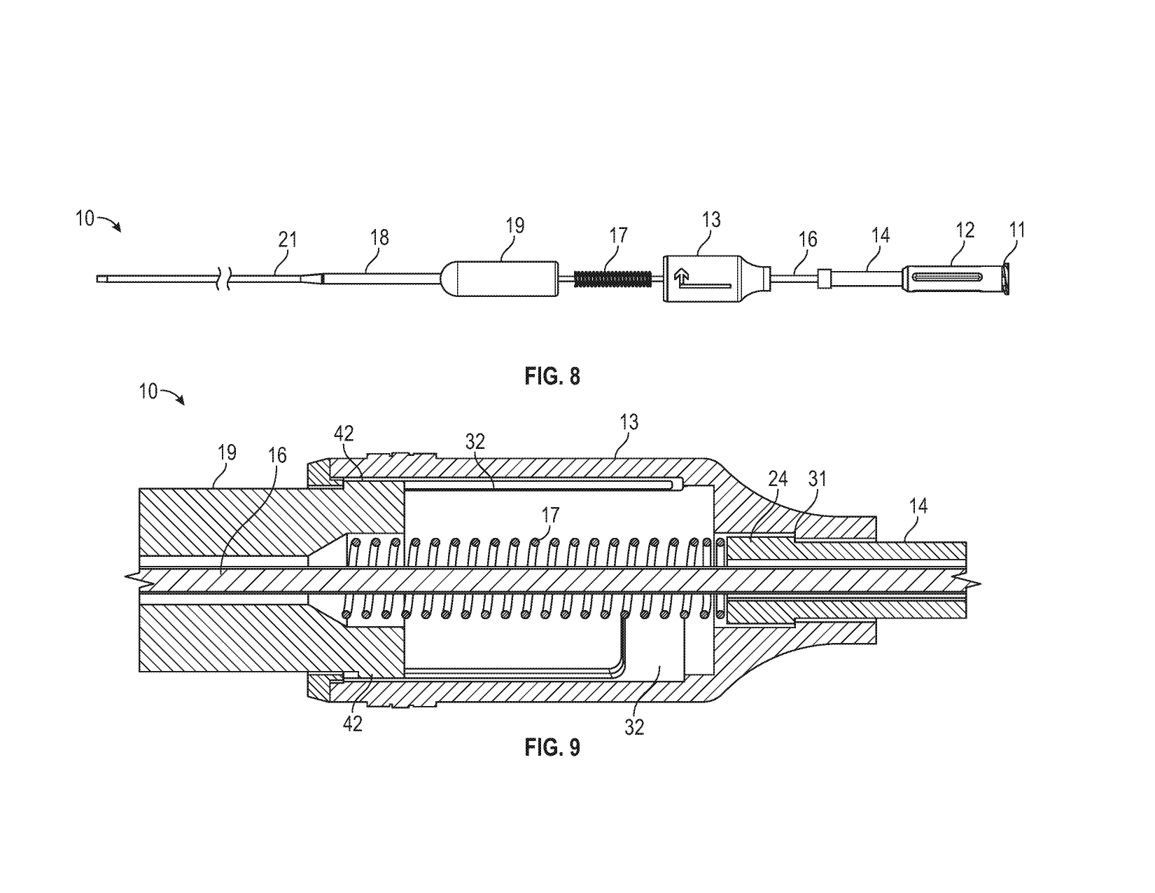

Referring now to FIGS. 8-14, a crossing device can provide a locking feature to maintain the plunger and/or the interventional tool thereof in a particular position (e.g., retracted, partially extended, and/or fully extended) with respect to other components of the crossing device. In some embodiments, as shown in FIG. 8, proximal handle 13 can be adjustable with respect to distal handle 19 during use of crossing device 10. It should be recognized that adjustment of proximal handle 13 with respect to distal handle 19 can include movement of proximal handle 13 with respect to distal handle 19, another item, and/or a location within an external frame of reference, for example while distal handle 19 is stationary. It should also be recognized that adjustment of proximal handle 13 with respect to distal handle 19 can include movement of distal handle 19 with respect to proximal handle 13, another item, and/or a location within the external frame of reference, for example while proximal handle 13 is stationary. It should also be recognized that adjustment of proximal handle 13 with respect to distal handle 19 can include simultaneous movement of both distal handle 19 and proximal handle 13. A selection of one of multiple configurations of proximal handle 13 with respect to distal handle 19 can define a limit on the extent of retraction available to plunger 14 under the influence of spring 17. As such, the operation of proximal handle 13 with respect to distal handle 19 can provide a variety of different characteristics and operations for other components of crossing device 10. It should be understood that the crossing devices and components thereof as illustrated in FIGS. 8-14 can include one or more of the features of the crossing devices and components thereof as illustrated in FIGS. 1-7. In some embodiments, such combinations of features are contemplated except where indicated otherwise.

In some embodiments, as shown in FIG. 9, spring 17 is disposed between at least a portion of distal handle 19 and at least a portion of plunger 14. For example, as shown in FIG. 6, spring 17 extends between plunger 14 and distal handle 19. In some embodiments, spring 17 applies a biasing force to urge distal handle 19 and plunger 14 away from each other. As further shown in FIG. 9, plunger 14 extends through at least a portion of proximal handle 13. In some embodiments, plunger 14 includes distal portion 24 that has an enlarged cross-sectional dimension relative to one or more other portions of plunger 14. In some embodiments, proximal handle 13 defines shoulder 31 for receiving distal portion 24. The engagement (e.g., abutting) between distal portion 24 of plunger 14 and shoulder 31 of proximal handle 13 prevent travel of plunger 14 with respect to proximal handle 13 beyond a certain point. As such, the biasing force provided by spring 17 and applied to plunger 14 can be transmitted to proximal handle 13, for example when distal portion 24 of plunger 14 abuts shoulder 31 of proximal handle 13. However, plunger 14 can be longitudinally advanced (e.g., extended and/or actuated) against the biasing force of spring 17 to a correspondingly advance interventional tool 16 with respect to distal handle 19. Such longitudinal advancement of plunger 14 may or may not be accompanied by longitudinal advancement of proximal handle 13. Plunger 14 can be permitted to rotate freely and/or within a range of motion with respect to proximal handle 13.

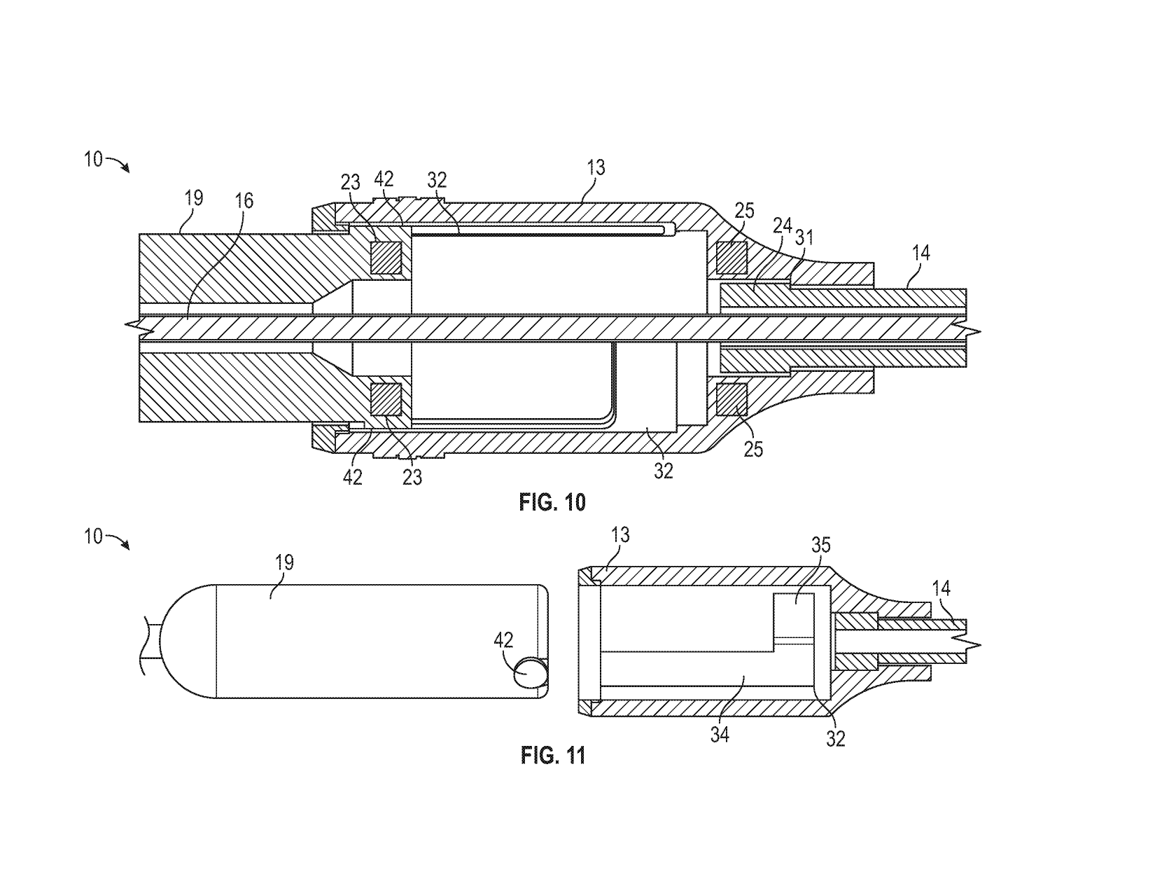

In some embodiments, as shown in FIG. 10, each of handle 19 and plunger 14 can include one or more magnets. For example, in addition to spring 17 and/or as an alternative to spring 17, one or more magnets 23 can be located at and/or coupled to handle 19, and one or more magnets 25 can be located at and/or coupled to plunger 14. In some embodiments, magnets 23 and 25 apply a biasing force to urge distal handle 19 and plunger 14 away from each other. For example, magnets 23 and 25 can be arranged to be magnetically repulsed away from each other. For another example, the magnets 23 and 25 can have common magnet poles (e.g., north or south) facing towards each other. The magnetic repulsive force can increase in magnitude as magnets 23 and 25 approach each other (e.g., as handle 19 and plunger 14 approach each other). In some embodiments, each of the magnets 23 and 25 can be formed as an array of magnets, a continuous magnet (e.g., a ring magnet), and/or as any other element configured to generate a magnetic field. In one or more aspects, the other features of the crossing device 10 can be the same, similar, or different from those described herein with respect to FIG. 9.

In some embodiments, as further shown in FIGS. 9 and 10, proximal handle 13 includes one or more channels 32, and distal handle 19 includes one or more engagers 42 for engaging a respective one of the one or more channels 32 for guiding motion of proximal handle 13 with respect to distal handle 19. The one or more channels 32 can include one or more recesses, cavities, bounded spaces, and the like. The one or more engagers 42 can include one or more protrusions, bumps, stubs, pins, and the like. While FIGS. 9 and 10 illustrate two engagers 42 and two channels 32, it should be recognized that proximal handle 13 can include any number of channels 32, and distal handle 19 can include any number of engagers 42. Each of engagers 42 can be arranged to engage a corresponding one of the one or more channels 32. As such, one or more engagers 42 and one or more channels 32 can have complementary shapes.

While channels 32 are illustrated as being defined by proximal handle 13 and engagers 42 are illustrated as being defined by distal handle 19, it should be recognized that any arrangement of engagers and channels can be provided. For example, one or more engagers 42 can be provided by proximal handle 13, and one or more channels 32 can be provided by distal handle 19.

In some embodiments, as shown in FIG. 11, channel 32 can include one or more portions that extend in different directions to provide various directions of travel for engager 42. For example, channel 32 can include longitudinal portion 34 and circumferential portion 35. Longitudinal portion 34 can extend longitudinally (e.g., parallel to a longitudinal or central axis of crossing device 10), and circumferential portion 35 can extend circumferentially (e.g., about the longitudinal or central axis of crossing device 10). As such, different motions of proximal handle 13 with respect to distal handle 19 may be required to effect travel of engager 42 within each of the different portions of channel 32.

In some embodiments, longitudinal portion 34 can extend to a distal end of proximal handle 13, such that engager 42 can be received by longitudinal motion of proximal handle 13 with respect to distal handle 19. Engager 42 can travel longitudinally within longitudinal portion 34 of channel 32 by translating proximal handle 13 longitudinally with respect to distal handle 19. It should be recognized that such longitudinal advancement of proximal handle 13 with respect to distal handle 19 can cause plunger 14 (and/or interventional tool 16) to correspondingly advance longitudinally.

In some embodiments, upon reaching a furthest extent of longitudinal extension within longitudinal portion 34, engager 42 can subsequently travel circumferentially within circumferential portion 35 of channel 32. Engager 42 can travel circumferentially within circumferential portion 35 of channel 32 by rotating proximal handle 13 with respect to distal handle 19. It should be recognized that, while engager 42 is located within circumferential portion 35 of channel 32, longitudinal retraction of proximal handle 13 with respect to distal handle 19 can be limited and/or prevented. For example, a side of circumferential portion 35 of channel 32 (e.g., facing distal handle 19) can provide a structure that prevents engager 42 from moving longitudinally until engager 42 returns to longitudinal portion 34 of channel 32 (e.g., by reverse rotation of proximal handle 13 with respect to distal handle 19). Such an engagement can be maintained against the biasing force of the spring (not shown in FIG. 11). In some embodiments, the biasing force of the spring can enhance engagement of engager 42 within circumferential portion 35 of channel 32. Such engagement can be maintained until proximal handle 13 is rotated with respect to distal handle 19 and engager 42 is returned to longitudinal portion 34 of channel 32.

It should be recognized that, while engagers and channels are illustrated with a particular shape, size, and/or geometry, and engagement between proximal handle 13 and distal handle 19 can involve one or more of a variety of features. For example, proximal handle 13 and distal handle 19 can selectably (e.g., releasably) engaged with one or more threads, latches, locks, pins, magnets, and the like.

It should be recognized that, while proximal handle 13 is in a locked configuration with respect to distal handle 19 (e.g., with engager 42 within circumferential portion 35 of channel 32), plunger 14 may be limited in its available extent of proximal retraction. However, plunger 14 (and/or interventional tool 16) may nonetheless be advanced distally while proximal handle 13 is in the locked configuration with respect to distal handle 19. Spring 17 may apply its biasing force during such advancement and retraction of plunger 14.

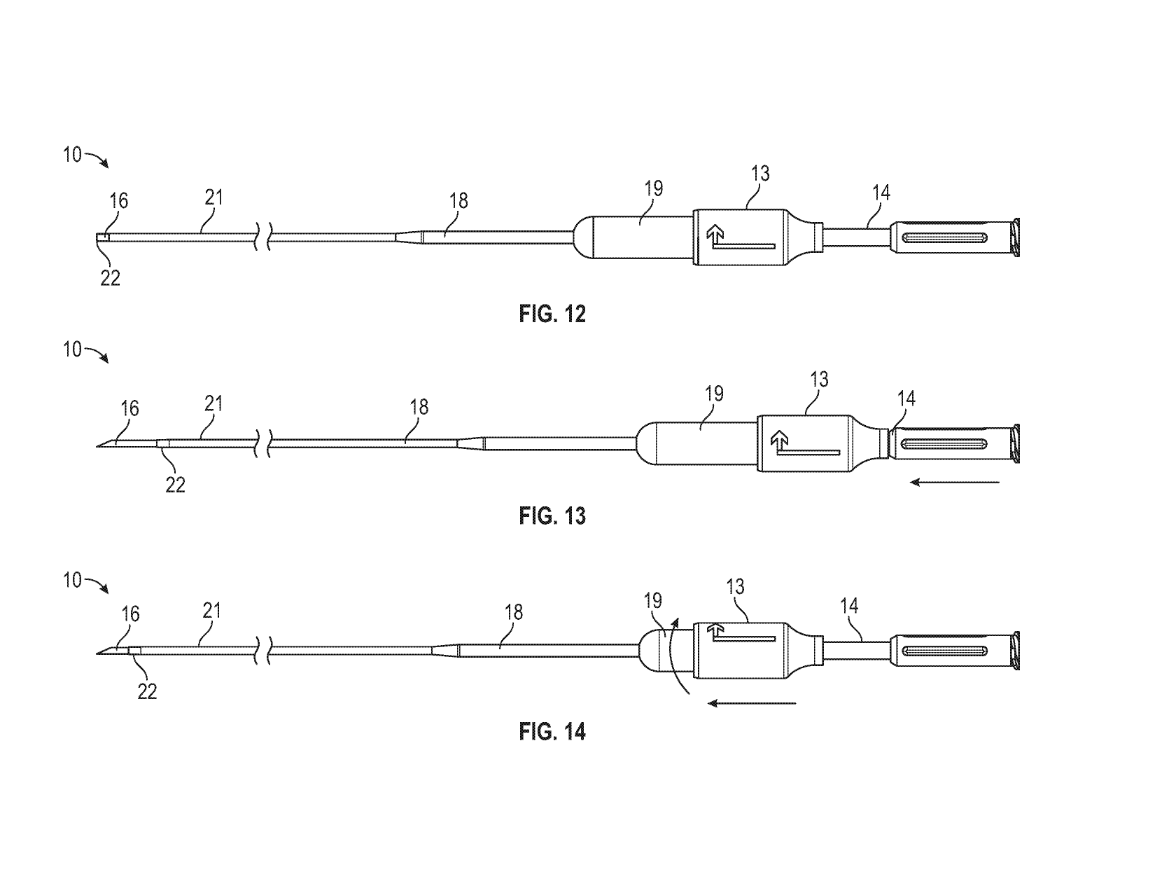

As shown in FIG. 12, crossing device 10 can be provided in a retracted configuration. For example, proximal handle 13 can be in a proximal position with respect to distal handle 19. By further example, plunger 14 can be located distally in a retracted (e.g., natural or relaxed) configuration. Accordingly, interventional tool 16 can be in a corresponding retracted (e.g., natural or relaxed) configuration, such as entirely within catheter 21. While in such a position, interventional tool 16 can be covered, rather than exposed, such that it does not act directly upon any anatomy in the vicinity thereof. Additionally or alternatively, while in such a position, interventional tool 16 can be retracted proximally with respect to distal end 22 of catheter 21, such that catheter 21 is provided with a length that is empty. At such a length, catheter 21 (e.g., at a distal end portion thereof) can be more flexible to facilitate travel of catheter 21 through tortuous anatomy.

As shown in FIG. 13, while proximal handle 13 is in a retracted configuration with respect to distal handle 19, plunger 14 may be provided with a range of motion, such as an extent of distal advancement. For example, plunger 14 can be advanced distally (e.g., until abutting proximal handle 13). Such advancement of plunger 14 can cause distal advancement of interventional tool 16, such as to extend beyond a distal end 22 of catheter 21. This can correspond to action of interventional tool 16 on an occlusion or other structure. Spring 17 may apply its biasing force during such advancement of plunger 14 to bias it to the retracted configuration of FIG. 12.

As shown in FIG. 14, crossing device 10 can be provided in a locked configuration. For example, proximal handle 13 can be moved to a distal position with respect to distal handle 19. This can be achieved with the translation and/or rotation described herein with respect to FIG. 11. Such engagement can be maintained until proximal handle 13 is reverse rotated with respect to distal handle 19 and allowed to move longitudinally. By further example, plunger 14 can be located distally in a retracted (e.g., natural or relaxed) configuration. However, unlike the retracted configuration of FIG. 12, the distal position and locked configuration of proximal handle 13 with respect to distal handle 19 causes plunger 14 and interventional tool 16 to have a more distally advanced configuration, even when not actuated. It should be recognized that, while proximal handle 13 is in a locked configuration with respect to distal handle 19, plunger 14 may be limited in its available extent of proximal retraction. As shown in FIG. 14, the position of interventional tool 16 can be distal to the distal end 22 of catheter 21. Plunger 14 and/or interventional tool 16 may also be advanced distally (not shown) while proximal handle 13 is in the locked configuration with respect to distal handle 19. Spring 17 may apply its biasing force during such advancement and retraction of plunger 14.

It should be understood that the locked configuration of proximal handle 13 and the accompanying configurations of plunger 14 and/or interventional tool 16 can correspond to other positions. For example, while proximal handle 13 is in a locked configuration with respect to distal handle 19, and plunger 14 is in a retracted configuration, interventional tool 16 can be entirely within catheter 21 (e.g., proximal to distal end 22 of catheter 21). By further example, while proximal handle 13 is in a locked configuration with respect to distal handle 19, and plunger 14 is in an extended configuration, interventional tool 16 can extend distally beyond distal and 22 of catheter 21.

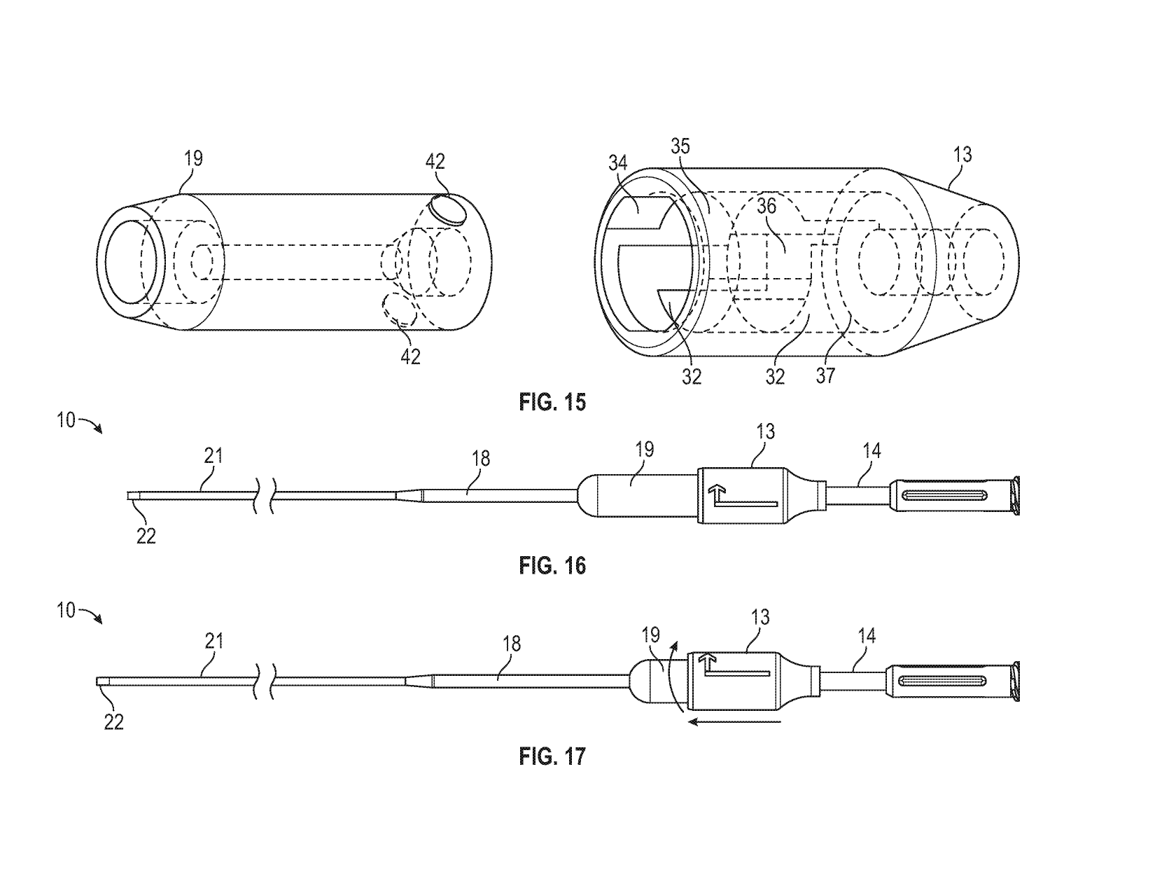

Referring now to FIGS. 15-18, a crossing device can provide a locking feature to maintain the plunger and/or the interventional tool thereof in one of multiple positions (e.g., retracted, partially extended, and/or extended) with respect to other components of the crossing device. In some embodiments, as shown in FIG. 15, proximal handle 13 can be adjustable with respect to distal handle 19 during use of crossing device 10. It should be recognized that adjustment of proximal handle 13 with respect to distal handle 19 can include movement of proximal handle 13 with respect to distal handle 19, another item, and/or a location within an external frame of reference, for example while distal handle 19 is stationary. It should also be recognized that adjustment of proximal handle 13 with respect to distal handle 19 can include movement of distal handle 19 with respect to proximal handle 13, another item, and/or a location within the external frame of reference, for example while proximal handle 13 is stationary. It should also be recognized that adjustment of proximal handle 13 with respect to distal handle 19 can include simultaneous movement of both distal handle 19 and proximal handle 13. As such, the operation of proximal handle 13 with respect to distal handle 19 can provide a variety of different characteristics and operations for other components of crossing device 10. It should be understood that the crossing devices and components thereof as illustrated in FIGS. 15-18 can include one or more of the features of the crossing devices and components thereof as illustrated in FIGS. 1-14. In some embodiments, such combinations of features are contemplated except where indicated otherwise.

In some embodiments, as further shown in FIG. 15, proximal handle 13 includes one or more channels 32, and distal handle 19 includes one or more engagers 42 for engaging a respective one of the one or more channels 32 for guiding motion of proximal handle 13 with respect to distal handle 19. The one or more channels 32 can include one or more recesses, cavities, bounded spaces, and the like. The one or more engagers 42 can include one or more protrusions, bumps, stubs, pins, and the like. While FIG. 15 illustrates two engagers 42 and two channels 32, it should be recognized that proximal handle 13 can include any number of channels 32, and distal handle 19 can include any number of engagers 42. Each of engagers 42 can be arranged to engage a corresponding one of the one or more channels 32. As such, one or more engagers 42 and one or more channels 32 can have complementary shapes.

While channels 32 are illustrated as being defined by proximal handle 13 and engagers 42 are illustrated as being defined by distal handle 19, it should be recognized that any arrangement of engagers and channels can be provided. For example, one or more engagers 42 can be provided by proximal handle 13, and one or more channels 32 can be provided by distal handle 19.

In some embodiments, as shown in FIG. 15, channel 32 can include one or more portions that extend in different directions to provide various directions of travel for engager 42. For example, channel 32 can include first longitudinal portion 34, first circumferential portion 35, second longitudinal portion 36, and second circumferential portion 37. First longitudinal portion 34 and/or second longitudinal portion 36 can extend longitudinally (e.g., parallel to a longitudinal or central axis of crossing device 10), and first circumferential portion 35 and/or second circumferential portion 37 can extend circumferentially (e.g., about the longitudinal or central axis of crossing device 10). As such, different motions of proximal handle 13 with respect to distal handle 19 may be required to effect travel of engager 42 within any two or more of the different portions of channel 32.

In some embodiments, first longitudinal portion 34 can extend to a distal end of proximal handle 13, such that engager 42 can be received by longitudinal motion of proximal handle 13 with respect to distal handle 19. Engager 42 can travel longitudinally within first longitudinal portion 34 of channel 32 by translating proximal handle 13 longitudinally with respect to distal handle 19. It should be recognized that such longitudinal advancement of proximal handle 13 with respect to distal handle 19 can cause plunger 14 (and/or interventional tool 16) to correspondingly advance longitudinally (e.g., to an intermediate position).

In some embodiments, upon reaching a furthest extent of longitudinal extension within first longitudinal portion 34, engager 42 can subsequently travel circumferentially within first circumferential portion 35 of channel 32. Engager 42 can travel circumferentially within first circumferential portion 35 of channel 32 by rotating proximal handle 13 with respect to distal handle 19. It should be recognized that, while engager 42 is located within first circumferential portion 35 of channel 32, longitudinal retraction of proximal handle 13 with respect to distal handle 19 can be limited and/or prevented. For example, a side of first circumferential portion 35 of channel 32 (e.g., facing distal handle 19) can provide a structure that prevents engager 42 from moving longitudinally until engager 42 returns to first longitudinal portion 34 of channel 32 (e.g., by reverse rotation of proximal handle 13 with respect to distal handle 19). Such an engagement can be maintained against the biasing force of the spring (not shown in FIG. 15). In some embodiments, the biasing force of the spring can enhance engagement of engager 42 within first circumferential portion 35 of channel 32. Such engagement can be maintained until proximal handle 13 is rotated with respect to distal handle 19 and engager 42 is returned to first longitudinal portion 34 of channel 32.

In some embodiments, second longitudinal portion 36 can extend to first circumferential portion 35, such that engager 42 can be received by further longitudinal motion of proximal handle 13 with respect to distal handle 19 while engager 42 is within a particular portion of first circumferential portion 35. Engager 42 can further travel longitudinally within second longitudinal portion 36 of channel 32 by translating proximal handle 13 longitudinally with respect to distal handle 19. It should be recognized that such longitudinal advancement of proximal handle 13 with respect to distal handle 19 can cause plunger 14 (and/or interventional tool 16) to correspondingly advance longitudinally (e.g., to an advanced position distal to the intermediate position).

In some embodiments, upon reaching a furthest extent of longitudinal extension within second longitudinal portion 36, engager 42 can subsequently further travel circumferentially within second circumferential portion 37 of channel 32. Engager 42 can travel circumferentially within second circumferential portion 37 of channel 32 by further rotating proximal handle 13 with respect to distal handle 19. It should be recognized that, while engager 42 is located within second circumferential portion 37 of channel 32, longitudinal retraction of proximal handle 13 with respect to distal handle 19 can be limited and/or prevented. For example, a side of second circumferential portion 37 of channel 32 (e.g., facing distal handle 19) can provide a structure that prevents engager 42 from moving longitudinally until engager 42 returns to second longitudinal portion 36 of channel 32 (e.g., by reverse rotation of proximal handle 13 with respect to distal handle 19). Such an engagement can be maintained against the biasing force of the spring (not shown in FIG. 15). In some embodiments, the biasing force of the spring can enhance engagement of engager 42 within second circumferential portion 37 of channel 32. Such engagement can be maintained until proximal handle 13 is rotated with respect to distal handle 19 and engager 42 is returned to second longitudinal portion 36, first circumferential portion 35, and/or first longitudinal portion 34 of channel 32.

As shown in FIG. 16, crossing device 10 can be provided in a retracted configuration. For example, proximal handle 13 can be in a proximal position with respect to distal handle 19. By further example, plunger 14 can be located distally in a retracted (e.g., natural or relaxed) configuration. Accordingly, interventional tool 16 can be in a corresponding retracted (e.g., natural or relaxed) configuration, such as entirely within catheter 21. While in such a position, interventional tool 16 can be covered, rather than exposed, such that it does not act directly upon any anatomy in the vicinity thereof. Additionally or alternatively, while in such a position, interventional tool 16 can be retracted proximally with respect to distal end 22 of catheter 21, such that catheter 21 is provided with a length that is empty. At such a length, catheter 21 (e.g., at a distal end portion thereof) can be more flexible to facilitate travel of catheter 21 through tortuous anatomy.

As shown in FIG. 17, crossing device 10 can be provided in a first locked configuration. For example, proximal handle 13 can be moved to an intermediate position with respect to distal handle 19. This can be achieved with the translation and/or rotation described herein with respect to FIG. 15. Such engagement can be maintained until proximal handle 13 is reverse rotated with respect to distal handle 19 and allowed to move longitudinally. By further example, plunger 14 can be located distally in a retracted (e.g., natural or relaxed) configuration. However, the distal position and first locked configuration of proximal handle 13 with respect to distal handle 19 causes plunger 14 and interventional tool 16 to have a more distally advanced configuration, even when not actuated. As shown in FIG. 17, the position of interventional tool 16 can be proximal to the distal end 22 of catheter 21. In some embodiments, while proximal handle 13 is in the first locked configuration with respect to distal handle 19, plunger 14 may be provided with a range of motion, such as an extent of distal advancement. For example, plunger 14 can be advanced distally (e.g., until abutting proximal handle 13). Such advancement of plunger 14 can cause distal advancement of interventional tool 16, such as to extend beyond a distal end 22 of catheter 21. This can correspond to action of interventional tool 16 on an occlusion or other structure. Spring 17 may apply its biasing force during such advancement and retraction of plunger 14.

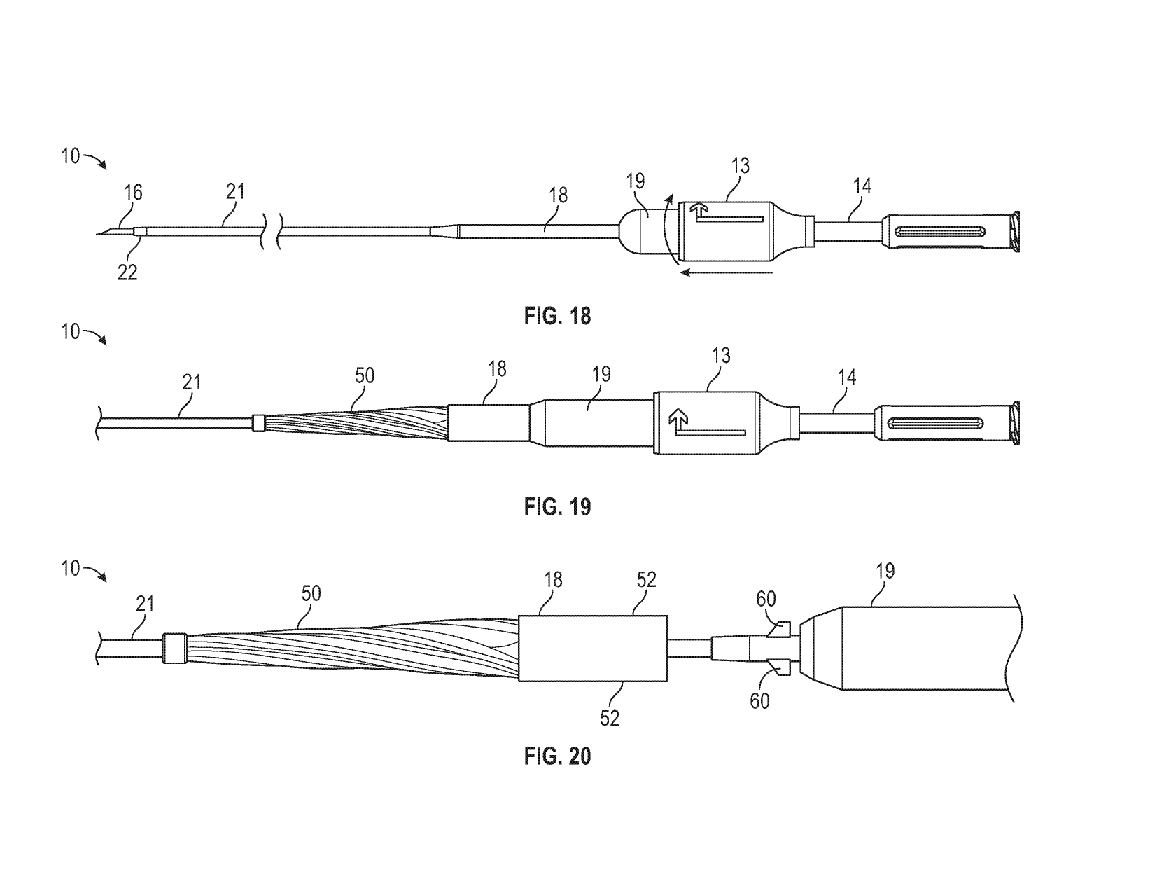

As shown in FIG. 18, crossing device 10 can be provided in a second locked configuration. For example, proximal handle 13 can be moved to a distal position with respect to distal handle 19. This can be achieved with the translation and/or rotation described herein with respect to FIG. 15. Such engagement can be maintained until proximal handle 13 is reverse rotated with respect to distal handle 19 and allowed to move longitudinally. By further example, plunger 14 can be located distally in a retracted (e.g., natural or relaxed) configuration. However, the distal position and second locked configuration of proximal handle 13 with respect to distal handle 19 causes plunger 14 and interventional tool 16 to have a more distally advanced configuration, even when not actuated. As shown in FIG. 18, the position of interventional tool 16 can be distal to the distal end 22 of catheter 21. In some embodiments, while proximal handle 13 is in the second locked configuration with respect to distal handle 19, plunger 14 may be provided with a range of motion, such as an extent of distal advancement. For example, plunger 14 can be advanced distally (e.g., until abutting proximal handle 13). Such advancement of plunger 14 can cause distal advancement of interventional tool 16, such as to extend further beyond a distal end 22 of catheter 21. This can correspond to action of interventional tool 16 on an occlusion or other structure. Spring 17 may apply its biasing force during such advancement and retraction of plunger 14.

It should be understood that the locked configurations of proximal handle 13 and the accompanying configurations of plunger 14 and/or interventional tool 16 can correspond to other positions. For example, while proximal handle 13 is in a first and/or second locked configuration with respect to distal handle 19, and plunger 14 is in a retracted configuration, interventional tool 16 can be entirely within catheter 21 (e.g., proximal to distal end 22 of catheter 21). By further example, while proximal handle 13 is in a first and/or second locked configuration with respect to distal handle 19, and plunger 14 is in a retracted configuration, interventional tool 16 can extend distally beyond distal and 22 of catheter 21. By further example, while proximal handle 13 is in a first and/or second locked configuration with respect to distal handle 19, and plunger 14 is in an extended configuration, interventional tool 16 can extend distally beyond distal and 22 of catheter 21.

Accordingly, by providing multiple longitudinal channels and/or circumferential channels, an engager can travel to different portions to provide multiple locked configurations. It should be recognized that each locked configuration can correspond to a particular position and/or range of motion for an interventional tool based on actuation of a plunger. It should be further recognized that the position and/or ranges of motion for the interventional tool can be selected based on selection of the corresponding channels and/or other features of the handles.

Referring now to FIGS. 19 and 20, a crossing device can include one or more strain relief features. In some embodiments, as shown in FIG. 19, outer body 18 can include and/or be connected to strain relief body 50. Strain relief body 50 can extend from distal handle 19 along at least a length of catheter 21. Strain relief body 50 can prevent non-uniform curvature, i.e., “kinking” of catheter 21 at or near the junction with outer body 18. Strain relief body 50 can include a material is more flexible than the outer body 18 and/or less flexible than catheter 21. Strain relief body 50 can distribute bending forces along its length and away from the junction of catheter 21 and outer body 18. Strain relief body 50 can include one or more fins and/or other structures that extend, for example, in a helical and/or spiral shape along a longitudinal length of strain relief body 50.

In some embodiments, as shown in FIG. 20, strain relief body 50 can include one or more strain relief engagers 52 for being engaged by one or handle engagers 60. For example, as further shown in FIG. 20, handle engagers 60 can include one or more protrusions extending radially. Each of handle engagers 60 can extend into a respective one of strain relief engagers 52 of strain relief body 50. Engagement between handle engagers 60 and strain relief engagers 52 can secure strain relief body 50 in an axial position with respect to distal handle 19. Additionally or alternatively, engagement between handle engagers 60 and strain relief engagers 52 can transmit torque between distal handle 19 and strain relief body 50.

In some embodiments, a kit of parts is disclosed. One or more kits of parts can be envisioned by the person skilled in the art, the kits of parts including at least one component disclose herein and configured to perform at least one of the methods herein disclosed. Likewise, directions for use (“DFU”) are included and the device may be part of a surgical tray or other packaged accessory set for surgeries. The kit may be a sub-component of a surgical tray.

Various examples of aspects of the disclosure are described below as clauses for convenience. These are provided as examples, and do not limit the subject technology.

Clause A: a crossing device comprising: a catheter having a distal handle defining an engager; a proximal handle defining a channel having a longitudinal portion and a circumferential portion, wherein the proximal handle is configured to translate along a longitudinal axis with the engager within the longitudinal portion, wherein the proximal handle is further configured to rotate about the longitudinal axis with the engager within the circumferential portion; an interventional tool extending within the catheter and the proximal handle; and a plunger coupled to a proximal end of the interventional tool, wherein the plunger is configured to move between the proximal handle and the distal handle.

Clause B: a crossing device comprising: a catheter having a distal handle; a proximal handle, wherein the proximal handle is configured to translate along a longitudinal axis from a retracted configuration of the proximal handle and rotate about the longitudinal axis to a locked configuration of the proximal handle with respect to the distal handle; an interventional tool extending within the catheter and the proximal handle; and a plunger coupled to a proximal end of the interventional tool, wherein: the plunger is configured to move within a first range of motion between the proximal handle and the distal handle while the proximal handle is in the retracted configuration; and the plunger is configured to move within a second range of motion, different from the first range of motion, between the proximal handle and the distal handle while the proximal handle is in the locked configuration.

Clause C: a method comprising: providing a crossing device with an interventional tool within a catheter while an engager of a distal handle of the catheter is within a longitudinal portion of a channel of a proximal handle such that a plunger coupled to the interventional tool has a first range of motion with respect to the catheter; advancing the proximal handle distally with respect to the distal handle by advancing the engager within the longitudinal portion of the channel such that the plunger has a second range of motion, different from the first range of motion, with respect to the catheter; and rotating the proximal handle with respect to the distal handle by moving the engager out of the longitudinal portion and circumferentially within a circumferential portion of the channel such that the proximal handle is in a locked configuration with respect to the distal handle in which longitudinal retraction of the proximal handle is limited.

One or more of the above clauses can include one or more of the features described below. It is noted that any of the following clauses may be combined in any combination with each other, and placed into a respective independent clause, e.g., clause A, B, or C.

Clause 1: a spring biasing the plunger and the distal handle away from each other.

Clause 2: the plunger includes a first magnet; the distal handle includes a second magnet.

Clause 3: the first magnet and the second magnet are configured to bias the plunger and the distal handle away from each other.

Clause 4: the circumferential portion of the channel is proximal to the longitudinal portion of the channel.

Clause 5: in a retracted configuration of the plunger and a retracted configuration of the proximal handle with the engager within the longitudinal portion of the channel, a distal end of the interventional tool is proximal to a distal end of the catheter; in the retracted configuration of the plunger and a locked configuration of the proximal handle with the engager within the circumferential portion of the channel, the distal end of the interventional tool is distal to the distal end of the catheter; and in an extended configuration of the plunger and the locked configuration of the proximal handle with the engager within the circumferential portion of the channel, the distal end of the interventional tool is distal to the distal end of the catheter.

Clause 6: in a retracted configuration of the plunger and a retracted configuration of the proximal handle with the engager within the longitudinal portion of the channel, a distal end of the interventional tool is proximal to a distal end of the catheter; in the retracted configuration of the plunger and a locked configuration of the proximal handle with the engager within the circumferential portion of the channel, the distal end of the interventional tool is proximal to the distal end of the catheter; and in an extended configuration of the plunger and the locked configuration of the proximal handle with the engager within the circumferential portion of the channel, the distal end of the interventional tool is distal to the distal end of the catheter.

Clause 7: the engager is a first engager; the channel is a first channel; the distal handle further defines a second engager; and the proximal handle further defines a second channel for receiving the second engager.

Clause 8: the longitudinal portion is a first longitudinal portion; the circumferential portion is a first circumferential portion; the channel has a second longitudinal portion and a second circumferential portion; and the proximal handle is further configured to translate along the longitudinal axis with the engager within the second longitudinal portion, wherein the proximal handle is further configured to rotate about the longitudinal axis with the engager within the second circumferential portion.

Clause 9: the first longitudinal portion of the channel is distal to the first circumferential portion of the channel; the first circumferential portion of the channel is distal to the second longitudinal portion of the channel; and the second longitudinal portion of the channel is distal to the second circumferential portion of the channel.

Clause 10: in a retracted configuration of the plunger and a retracted configuration of the proximal handle with the engager within the first longitudinal portion of the channel, a distal end of the interventional tool is proximal to a distal end of the catheter; in the retracted configuration of the plunger and a first locked configuration of the proximal handle with the engager within the first circumferential portion of the channel, the distal end of the interventional tool is proximal to the distal end of the catheter; and in the retracted configuration of the plunger and a second locked configuration of the proximal handle with the engager within the second circumferential portion of the channel, the distal end of the interventional tool is distal to the distal end of the catheter.

Clause 11: in a retracted configuration of the plunger and the retracted configuration of the proximal handle, a distal end of the interventional tool is proximal to a distal end of the catheter; in the retracted configuration of the plunger and the locked configuration of the proximal handle, the distal end of the interventional tool is distal to the distal end of the catheter; and in an extended configuration of the plunger and the locked configuration of the proximal handle, the distal end of the interventional tool is distal to the distal end of the catheter.

Clause 12: the second range of motion is smaller than the first range of motion.

Clause 13: while the engager is within the circumferential portion of the channel, advancing the plunger and the interventional tool distally with respect to the proximal handle and the catheter.

Clause 14: rotating the proximal handle with respect to the distal handle by moving the engager circumferentially within the circumferential portion of the channel such that the proximal handle is in an unlocked configuration with respect to the distal handle.

Clause 15: retracting the proximal handle proximally with respect to the distal handle by retracting the engager within the longitudinal portion of the channel such that the plunger has the first range of motion.

Clause 16: advancing the proximal handle distally with respect to the distal handle includes moving the proximal handle against a biasing force from a spring between the proximal handling and the distal handle.

Clause 17: advancing the proximal handle distally with respect to the distal handle includes moving the proximal handle against a biasing force from two or more magnets positioned at the proximal handling and the distal handle.

A reference to an element in the singular is not intended to mean one and only one unless specifically so stated, but rather one or more. For example, "a" module may refer to one or more modules. An element proceeded by "a," "an," "the," or "said" does not, without further constraints, preclude the existence of additional same elements.

Headings and subheadings, if any, are used for convenience only and do not limit the invention. The word exemplary is used to mean serving as an example or illustration, To the extent that the term include, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrases may apply to all configurations, or one or more configurations. A disclosure relating to such phrases may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any present elements of the various steps, operations or processes in a sample order, and are not meant to be limited to the specific order or hierarchy presented, These may be performed in serial, linearly, in parallel or in different order. It should be understood that the described instructions, operations, and systems can generally be integrated together in a single software/hardware product or packaged into multiple software/hardware products.

In one aspect, a term coupled or the like may refer to being directly coupled. In another aspect, a term coupled or the like may refer to being indirectly coupled.

Terms such as top, bottom, front, rear, side, horizontal, vertical, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology and the subject technology, is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 USC §112, sixth paragraph, unless the element is expressly recited using the phrase “means for” or, in the case of a method claim the element is recited using the phrase “step for”.

The title, background, brief description of the drawings, abstract, and drawings are hereby incorporated into the disclosure, and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

What is claimed is:

1. A crossing device comprising:
a catheter having a distal handle defining an engager;
a proximal handle defining a channel having a longitudinal portion and a circumferential portion, wherein the proximal handle is configured to translate along a longitudinal axis with the engager within the longitudinal portion, wherein the proximal handle is further configured to rotate about the longitudinal axis with the engager within the circumferential portion;
an interventional tool extending within the catheter and the proximal handle; and
a plunger coupled to a proximal end of the interventional tool, wherein the plunger is configured to move between the proximal handle and the distal handle.

2. The crossing device of claim 1, further comprising a spring biasing the plunger and the distal handle away from each other.

3. The crossing device of claim 1, wherein:
the plunger includes a first magnet;
the distal handle includes a second magnet
the first magnet and the second magnet are configured to bias the plunger and the distal handle away from each other.

4. The crossing device of claim 1, wherein the circumferential portion of the channel is proximal to the longitudinal portion of the channel.

5. The crossing device of claim 1, wherein:
in a retracted configuration of the plunger and a retracted configuration of the proximal handle with the engager within the longitudinal portion of the channel, a distal end of the interventional tool is proximal to a distal end of the catheter;
in the retracted configuration of the plunger and a locked configuration of the proximal handle with the engager within the circumferential portion of the channel, the distal end of the interventional tool is distal to the distal end of the catheter; and
in an extended configuration of the plunger and the locked configuration of the proximal handle with the engager within the circumferential portion of the channel, the distal end of the interventional tool is distal to the distal end of the catheter.

6. The crossing device of claim 1, wherein:
in a retracted configuration of the plunger and a retracted configuration of the proximal handle with the engager within the longitudinal portion of the channel, a distal end of the interventional tool is proximal to a distal end of the catheter;
in the retracted configuration of the plunger and a locked configuration of the proximal handle with the engager within the circumferential portion of the channel, the distal end of the interventional tool is proximal to the distal end of the catheter; and
in an extended configuration of the plunger and the locked configuration of the proximal handle with the engager within the circumferential portion of the channel, the distal end of the interventional tool is distal to the distal end of the catheter.

7. The crossing device of claim 1, wherein:
the engager is a first engager;
the channel is a first channel;
the distal handle further defines a second engager; and
the proximal handle further defines a second channel for receiving the second engager.

8. The crossing device of claim 1, wherein:
the longitudinal portion is a first longitudinal portion;
the circumferential portion is a first circumferential portion;
the channel has a second longitudinal portion and a second circumferential portion; and
the proximal handle is further configured to translate along the longitudinal axis with the engager within the second longitudinal portion, wherein the proximal handle is further configured to rotate about the longitudinal axis with the engager within the second circumferential portion.

9. The crossing device of claim 8, wherein:
the first longitudinal portion of the channel is distal to the first circumferential portion of the channel;
the first circumferential portion of the channel is distal to the second longitudinal portion of the channel; and
the second longitudinal portion of the channel is distal to the second circumferential portion of the channel.

10. The crossing device of claim 8, wherein:
in a retracted configuration of the plunger and a retracted configuration of the proximal handle with the engager within the first longitudinal portion of the channel, a distal end of the interventional tool is proximal to a distal end of the catheter;
in the retracted configuration of the plunger and a first locked configuration of the proximal handle with the engager within the first circumferential portion of the channel, the distal end of the interventional tool is proximal to the distal end of the catheter; and
in the retracted configuration of the plunger and a second locked configuration of the proximal handle with the engager within the second circumferential portion of the channel, the distal end of the interventional tool is distal to the distal end of the catheter.

11. A crossing device comprising:
a catheter having a distal handle;
a proximal handle, wherein the proximal handle is configured to translate along a longitudinal axis from a retracted configuration of the proximal handle and rotate about the longitudinal axis to a locked configuration of the proximal handle with respect to the distal handle;
an interventional tool extending within the catheter and the proximal handle; and
a plunger coupled to a proximal end of the interventional tool, wherein:
the plunger is configured to move within a first range of motion between the proximal handle and the distal handle while the proximal handle is in the retracted configuration; and
the plunger is configured to move within a second range of motion, different from the first range of motion, between the proximal handle and the distal handle while the proximal handle is in the locked configuration.

12. The crossing device of claim 11, wherein:
in a retracted configuration of the plunger and the retracted configuration of the proximal handle, a distal end of the interventional tool is proximal to a distal end of the catheter;
in the retracted configuration of the plunger and the locked configuration of the proximal handle, the distal end of the interventional tool is distal to the distal end of the catheter; and
in an extended configuration of the plunger and the locked configuration of the proximal handle, the distal end of the interventional tool is distal to the distal end of the catheter.

13. The crossing device of claim 11, wherein the second range of motion is smaller than the first range of motion.

14. A method comprising:
providing a crossing device with an interventional tool within a catheter while an engager of a distal handle of the catheter is within a longitudinal portion of a channel of a proximal handle such that a plunger coupled to the interventional tool has a first range of motion with respect to the catheter;
advancing the proximal handle distally with respect to the distal handle by advancing the engager within the longitudinal portion of the channel such that the plunger has a second range of motion, different from the first range of motion, with respect to the catheter; and
rotating the proximal handle with respect to the distal handle by moving the engager out of the longitudinal portion and circumferentially within a circumferential portion of the channel such that the proximal handle is in a locked configuration with respect to the distal handle in which longitudinal retraction of the proximal handle is limited.

15. The method of claim 14, further comprising, while the engager is within the circumferential portion of the channel, advancing the plunger and the interventional tool distally with respect to the proximal handle and the catheter.

16. The method of claim 14, wherein the second range of motion is smaller than the first range of motion.

17. The method of claim 14, further comprising rotating the proximal handle with respect to the distal handle by moving the engager circumferentially within the circumferential portion of the channel such that the proximal handle is in an unlocked configuration with respect to the distal handle.

18. The method of claim 17, further comprising retracting the proximal handle proximally with respect to the distal handle by retracting the engager within the longitudinal portion of the channel such that the plunger has the first range of motion.

19. The method of claim 14, wherein advancing the proximal handle distally with respect to the distal handle includes moving the proximal handle against a biasing force from a spring between the proximal handling and the distal handle.

20. The method of claim 14, wherein advancing the proximal handle distally with respect to the distal handle includes moving the proximal handle against a biasing force from two or more magnets positioned at the proximal handling and the distal handle.
